(19) United States
(12) Patent Application Publication
(10) Pub. No.: US 2022/0265871 A1
(43) Pub. Date: Aug. 25, 2022

(54) Method of Treating Cancer by Intratumoral Deposition of Radioactive Microparticles

(71) Applicants: ADVANCED ACCELERATOR APPLICATIONS, SAINT GENIS POUILLY (FR); INSTITUT D'ENSEIGNEMENT SUPERIEUR ET DE RECHERCHE EN ALIMENTATION,SANTE ANIMAL SCIENCES AGRONOMIQUES, Marcy L'etoile (FR)

(72) Inventors: Ilyes Zahi, Nantes (FR); Valeria Muzio, Colleretto Giacosa (TO) (IT); Maurizio Mariani, Ivrea (TO) (IT); Jean-Philippe Meunier, Saint Genis Pouilly (FR); Mehrdad Khosh Nevis, Lyon (FR); Claude Carozzo, Dommartin (FR); Frédérique Ponce, Lyon (FR)

(21) Appl. No.: 17/615,941

(22) PCT Filed: Jun. 15, 2020
(86) PCT No.: PCT/EP2020/066489

§ 371 Date: Dec. 2, 2021

(30) Foreign Application Priority Data
Jun. 14, 2019 (EP) 19305765.0

(51) Int. Cl. A61K 51/12; A61P 35/00

(52) U.S. Cl. CPC A61K 51/1251; A61P 35/00

(57) ABSTRACT

A composition of microparticles includes a non-radioactive and a radioactive isotope, and an injection vehicle, for use in the intratumoral treatment of cancer, by combined antitumoral effect of radioactivity and intratumoral microparticle deposition. The antitumoral effect provided by the cytotoxicity of radioactivity is enhanced by a modulation of the tumoral immune response induced by microparticle deposition inside the tumor, thereby decreasing the dose of radioactivity required to treat solid tumors. A method of treating solid tumors using a sub-optimal tumor-volume coverage dose of radioactivity combined with intratumoral microparticle deposition as a mean of tumor control is also disclosed.

### FIELD OF THE INVENTION

The present invention relates to methods of treating solid tumors by a combined antitumoral effect of radioactivity and in situ microparticle deposition, wherein the antitumoral effect provided by the cytotoxicity of radioactivity is enhanced by a modulation of the tumoral immune response induced by microparticle deposition inside the tumor, thereby decreasing the dose of radioactivity required to treat solid tumors. The present invention relates to a method of treating solid tumors using a sub-optimal tumor-volume coverage dose of radioactivity combined with in situ microparticle deposition as a mean of tumor control.

Malignant brain tumors and brain metastases are often considered fatal in adults because of extremely poor prognosis and frequent tumor recurrence. Glioblastoma is the most common primary brain tumor in adults. In addition to glioblastoma, brain metastasis from other primary tumors including those from the lung, skin, and breast cancers also represent significant number of the central nervous system (CNS) tumors. It has been reported that 15-30% of the patients with metastatic breast and lung cancers develop brain metastases. Radiation therapy (RT) is the front-line treatment for brain tumors and metastases. Even with treatments involving surgical intervention, radiation therapy (RT), and chemotherapy only a fraction of these patients with malignant brain tumor/metastasis survives longer than 2 years after diagnosis. The radio-resistance of glioblastoma is a great problem that makes the complete treatment impossible with current therapeutic methods.

Solid cancers develop in complex microenvironments (tumor stroma) comprising non-neoplastic cells (tumor stromal cells) that dramatically influence tumor-growth, transformation and metastasis. Among these non-neoplastic cells, Tumor-associated macrophages (TAMs) which are classified into M1 phenotype that suppress cancer progression and M2 phenotype that promote it, are important regulators of tumorigenesis. This is particular true in the case of most common brain tumors (glioma such as glioblastoma) for which a large portion of stromal cells are TAMs (macrophages/microglia). Glioblastoma infiltration is an obstacle to its complete resection and the presence of TAMs has an important role in resistance to radiotherapy. Therefore, combining radiotherapy with TAM targeting would be beneficial for the treatment of common brain tumors such as glioblastoma.

Brachytherapy is a type of internal radiotherapy in which radioactive implants are placed in or near a tumor. The direct intratumoral injection of radioactive microparticles (micro brachytherapy) is a promising therapeutic approach for cancer treatment. Holmium (Ho-165/Ho-166) microparticles, under various forms, have been more particular studied since holmium offers several advantages for therapy including its suitability for in vivo imaging. 165-Ho is stable, 166-Ho is a pure and high energy β− emitter (Emax=1.86 MeV, t1/2=26.8 h, γ-emission=80 keV 6.7%), with limited tissue penetration of the β-particles (8 mm maximum). 166-Ho is also a gamma ray emitter which makes it suitable for SPECT (Single Photon Emission Computed Tomography) imaging. Moreover, holmium is highly paramagnetic and has a high mass attenuation coefficient, making this atom visible on Magnetic Resonance Imaging (MRI) and Computed tomography (CT), respectively.

The therapeutic efficacy of intratumoral injection of holmium microparticles has been studied in various types of cancer. Ho-166 chitosan complex has been tested in a prostate cancer model in rats (Kwak, Cheol Hong et al., European Journal of Nuclear Medicine and Molecular Imaging 2005, 32, 1400-1405), in a phase IIb clinical trial of hepatocellular carcinomas (Kim et al., Clinical Cancer Research, 2006, 12, 543-548) and malignant glioma in rats (Ryoong Huh et al., Yonsei Med. J., 2005, 46(1), 51-60). Ho-166 Acetylacetonate microspheres (HoAcAcMS) have been tested in a renal carcinoma model (Butt et al., PLoS One. 8(1) 2013, e52178. doi: 10.1371), a mouse model for kidney cancer (W. Bult, Holmium microparticles for intratumoral radioablation, Thesis, University of Utrecht, 2010), oral squamous cell carcinoma in cats (Nimwegen et al., Vet. Comp. Oncol., 2017, DOI:10.1111/vco.12319) and head and neck squamous cell carcinoma (Bakker et al., Nuclear Medicine communications, 2018, 39, 213-221)

Promising results were observed. However, therapeutic effects were minimal, in particular for large tumors (human and domestic animal studies). This was explained by incomplete tumor coverage with effective radiation dose since it is recognized in the field (Barani et al. Current Understanding and Treatment of Gliomas pp 49-73—Springer Edition, 2015) that complete coverage of tumor-volume and surrounding infiltrative area with an effective radiation dose (considered to be >60 Gy to achieve the necrosis of the tumor) is essential to obtain a therapeutic effect. It was reported that technical difficulties in intratumoral administration of the microparticles resulted in incomplete tumor coverage with efficient radiation dose. Therefore, current researches aim to fulfill this recognized requirement of complete tumor coverage with effective radiation dose, for example, by improving microparticle administration techniques. There is also potential risk of toxicity with this technology since leakage of radioactive microparticles outside the tumor was frequently reported.

A more efficient method of microbrachytherapy combining therapeutic efficacy and lack of toxicity would be an advance for the treatment of solid tumors. Moreover, it would be desirable to have a method of microbrachytherapy that is effective in the treatment of tumors associated with pro-tumoral macrophage activation such as the main common brain tumors including glioblastoma. The aim of the present invention is to provide a method of microbrachytherapy which provides such advantages.

### SUMMARY OF THE INVENTION

The present invention relates to methods of treating solid tumors by a combined antitumoral effect of radioactivity and in situ microparticle deposition. The invention is based on the observation by the inventors that the antitumoral effect of microbrachytherapy provided by the cytotoxicity of radioactivity is enhanced by a modulation of the tumoral immune response induced by microparticle deposition inside the tumor. As a result of this enhancement, the dose of radioactivity required to treat solid tumors is reduced, allowing the use of sub-optimal tumor-volume coverage dose of radioactivity in cancer treatments, with a direct consequence of sparing the surrounding healthy tissue from radiation-induced toxicity. The inventors have studied the therapeutic effect of intratumoral injection of Ho-165/Ho-166 microparticles in a human glioblastoma minipig model. Surprisingly, they have found that sub-optimal tumor-volume coverage dose of radioactivity resulted in efficient tumor control and increased survival in the absence of visible toxic effects in the treated animals (example 6; FIG. 1, Tables 2, 3, 4). The methods comprise advantageously the intratumoral deposition of radioactive microparticles.

In particular, the invention relates to the use of a composition comprising microparticles comprising a radioactive isotope and possibly a non-radioactive isotope possibly from the same chemical element, and an injection vehicle, for the intratumoral treatment of cancer, by combined antitumoral effect of radioactivity and intratumoral microparticle deposition.

In some embodiments, intratumoral microparticle deposition induces a depletion of pro-tumoral macrophages (tumor-associated macrophages (TAMs) having a M2 phenotype or M2-TAMs) from the treated tumor.

In some embodiments, intratumoral microparticle deposition induces an anti-tumor immune response.

In some embodiments, the radioactive isotope is suitable for use in radiotherapy, in particular in internal radiotherapy. In some embodiments, the radioisotope is selected from the group comprising: holmium-166 (Ho-166 or ¹⁶⁶Ho), lutetium-177 (Lu-177 or ¹⁷⁷Lu), rhenium-186 or -188 (Re-186 (¹⁸⁶Re) or Re-188(¹⁸⁸Re)), gold-198 (Au-198 or ¹⁹⁸Au), yttrium-90 (Y-90 or ⁹⁰Y), thorium-227 (Th-227 or ²²⁷Th), actinium-225 (Ac-225 or ²²⁵Ac) and combinations thereof; preferably holmium-166. In some embodiments, the microparticles comprise holmium-165 and holmium-166. Preferably, the microparticles comprise at least 20% (w/w) of total holmium, more preferably at least 25% (w/w) of total holmium.

In some embodiments, the microparticles comprise or consist of polysiloxane, acetlyacetonate (AcAc), Poly L-Lactic Acid (PLLA), alginate, chitosan, resin, glass or combination thereof. In some preferred embodiments, the microparticles comprise or consist of polysiloxane.

In some embodiments, the microparticles, preferably Ho-165/Ho-166 microparticles such as Ho-165/Ho-166 polysiloxane microparticles, have a mean or median size from 0.1 μm to 10 μm, preferably 0.1 μm to 1 μm, more preferably 0.3 μm to 0.6 μm, even more preferably 0.4 μm to 0.5 μm.

In some embodiments, the microparticles, preferably Ho-165/Ho-166 microparticles such as Ho-165/Ho-166 polysiloxane microparticles, have a specific activity of at least 1 megabecquerel (MBq) per milligram, for example 1 to 10 MBq per milligram, such as in particular 2 to 3 MBq per milligram.

In some embodiments, the composition comprises a microparticle suspension, preferably Ho-165/Ho-166 microparticle suspension, such as Ho-165/Ho-166 polysiloxane microparticle suspension, having a density of at least 1 g/mL, for example 1 g/mL to 1.5 g/mL.

In some embodiments, the composition comprises a microparticle suspension, preferably Ho-165/Ho-166 microparticle suspension, such as Ho-165/Ho-166 polysiloxane microparticle suspension, having a viscosity of at least 3 mPa·s, preferably from 3 mPa·s to 12 mPa·s, more preferably 6 mPa·s to 10 mPa·s.

In some embodiments, the composition comprises an aqueous suspension of the microparticles, preferably Ho-165/Ho-166 microparticles, such as Ho-165/Ho-166 polysiloxane microparticles, in an injection vehicle comprising water and eventually low amount of ethanol, preferably less than 5% (v/v).

In some embodiments, the use of the composition comprises the intratumoral administration of a sub-optimal tumor-volume coverage dose of radioactivity. In some preferred embodiments, the use of the composition comprises the intratumoral administration of an absorbed dose superior or equal to 100 Grays in less than 95%, 85%, 75%, 65%, 55%, 45 or 35% of the tumor volume, preferably in 35% to 55% of the tumor volume. In other preferred embodiments, the use of the composition comprises the intratumoral administration of an absorbed dose superior or equal to 60 Grays in less than 95%, 85%, 75%, 65%, 55% or 35% of the tumor volume, preferably in 35% to 55% of the tumor volume.

In some embodiments, the cancer is selected from the group comprising: bladder, brain, breast, colon, gastric, eye, head and neck, skin, kidney, liver, lung, ovary, oral, prostate, small intestine, soft-tissue, testis, thyroid, and uterus cancer. Preferably, the cancer is brain cancer, in particular glioblastoma.

### DETAILED DESCRIPTION OF THE INVENTION

The present invention relates to methods of treating solid tumors by a combined antitumoral effect of radioactivity and in situ microparticle deposition. The combined antitumoral effect allows the use of sub-optimal tumor-volume coverage dose of radioactivity for treating solid tumors, with a direct consequence of sparing the surrounding healthy tissue from radiation-induced toxicity. The methods comprise advantageously the intratumoral deposition of radioactive microparticles.

As used herein, the term “microparticles” or “microspheres” refers to particles of any material(s) onto which isotopes (radioactive and non-radioactive) can be bound to produce particles loaded or doped with the radioactive and non-radioactive isotopes. The microparticles according to the invention are biocompatible and biopersistent.

As used herein, the term “radioactive microparticles” refers to microparticles comprising a non-radioactive and a radioactive isotope as defined in the present invention, unless the context clearly indicates otherwise.

By “biocompatible” is meant not toxic to the body, pharmaceutically acceptable.

By “biopersistent” is meant a material that is retained at the injection site when injected into a tissue or organ such as a tumor of an individual, and remains at the injection site at least for the period of therapy. The biopersistence includes the biodurability which is the stability or resistance to chemical processes in vivo such as dissolution or leaching. The biopersistence includes also the stability or resistance to physical/mechanical processes in vivo such as translocation, diffusion, splitting or breaking.

As used herein, the term “microparticle size” refers to the mean, median or mode value reflecting the particle size distribution by number, surface, volume, or frequency as determined by conventional particle size measuring techniques known to those skilled in the art, such as laser diffraction, dynamic light scattering, photon correlation spectroscopy, sedimentation field flow fractionation, disk centrifugation or electrical sensing methods. The size value may also include the distribution widths (standard deviation, variance).

As used herein, the term “patient”, “individual” or subject denotes a mammal, including humans, domestic animals such as for example bovine (cows and the like), ovine (sheep and the like), caprine (goats and the like) and equine (horses and the like), and commercial animals such as with no limitations dogs and cats. The patient is preferably a human.

As used herein, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

As used herein, the term “tumor” refers to a cancerous tumor including primary tumor and metastasis.

As used herein, the term “cancer” refers to any type of solid cancer, also designated as solid tumor, solid tumor cancer, or solid cancer tumor.

As used herein, the expression, “sub-optimal tumor-volume coverage dose of radioactivity” refers to a dose of radioactivity that is inferior to the estimated minimum absorbed dose to achieve the necrosis of the tumor to be treated (i.e., the effective radiation dose). The estimated minimum absorbed dose is 60 Grays to 100 Grays in the entire volume (100%) of the tumor.

As used herein, the terms “a”, “an”, and “the” include plural referents, unless the context clearly indicates otherwise. For example, “a radioactive isotope” as used herein is understood to represent one or more radioactive isotopes. As such, the term “a” (or “an”), “one or more” or “at least one” can be used interchangeably herein.

In particular, the invention relates to the use of a composition comprising microparticles comprising a non-radioactive and a radioactive isotope, and an injection vehicle, for the intratumoral treatment of cancer, by combined antitumoral effect of radioactivity and intratumoral deposition of microparticles.

Radioactivity destroys tumor cells by delivering a local high-energy emission. The direct cytotoxic effect of radioactivity on tumor cells is used for treating solid tumors. The effect of radioactivity is considered an alternative mean of physical ablation, similarly to surgical resection. In the present invention, local microparticle deposition inside the tumor provides an additional anti-tumoral effect to the direct cytotoxic effect of radioactivity by inducing a modulation of the tumoral immune response. Therefore, the overall anti-tumoral effect is enhanced using the method of the invention. As a result of this enhancement, the dose of radioactivity required to treat solid tumors is reduced, allowing the use of sub-optimal tumor-volume coverage dose of radioactivity in cancer treatments, with a direct consequence of sparing the surrounding healthy tissue from radiation-induced toxicity.

In some embodiments, intratumoral microparticle deposition induces a depletion of pro-tumoral macrophages (M2-TAMs) from the treated tumor. The pro-inflammatory macrophages (M2-TAMs) present into the tumor microenvironment are attracted by the non radioactive/radioactive microparticles and eliminated by a direct killing effect. This action is persistent over time beyond the radiation effect. The application of radioactivity inducing a local tumor-killing effect through physical means is combined with the depletion of pro-tumoral macrophages from the treated tumor induced by local (in situ) microparticle deposition.

In some embodiments, intratumoral microparticle deposition induces an anti-tumor immune response. In particular, the ionizing radiations delivered by the microparticles induce anti-tumor immunity mechanisms by the recruitment of external macrophages and/or dendritic cells and/or activation of T lymphocyte production. By this way, the microenvironment of the tumor is drastically changed and act in combination and in a synergetic manner with the direct cytotoxic mechanism of the radioactivity on the tumors cells. More specifically, in the present invention the local deposition of said radioactive microparticles exert a combined antitumoral effect of local tumor-killing through physical means (radioactivity), depletion of pro-tumoral macrophages (TAMs) from the treated tumor and anti-tumor immune cells recruitment activation.

The present invention can be considered part of the emerging field of Immuno-Oncology (I-O) therapy that utilizes the body's own immune system to fight diseases. The goal of I-O therapy is to restore the ability of the immune system to eliminate cancer cells by either activating the immune system directly, by inhibiting mechanisms of suppression by tumors, or by modulating/suppressing those mechanisms that favor tumor growth and aggressiveness. In addition to the effect of radioactivity on the tumor and macrophages, the microparticles used in the present invention exert, surprisingly, a direct killing effect on the tumor-associated macrophages (TAMs), which is prolonged due to the persistency of the microparticles, as stable implants in the tumor tissue. This effect is persistent over time, beyond the temporary effect induced by radiation. This elicits a continuous recruitment and direct toxic effect on TAMs over time, ensuring a prolonged anti-tumoral effect. These recruitments participate to the tumor shrinkage and the induction of anti-tumor immunity.

Thus the present invention relates to a novel combination of temporary suboptimal killing radioactivity exposure and in situ persistence of anti-TAM microparticles, which synergistically control cancer cell growth, limiting the sequelae of radiotherapy ablation of healthy tissue induced by current radiotherapy approaches. Said combination is able to induce an extended anti-tumor response in addition to its ability to disrupt tumor cell cycling through delivery of ionizing radiation. For example, it was observed that treatment with Ho-166 microparticles in human xenograft glioblastoma pig tumor model results in increased survival in the absence of any visible detrimental effect on the treated animals. In addition to the local internal radiotherapeutic effect, due to the in situ deposition, the radioactive particles according to the invention, such a Ho-166/Ho-165 microparticles allow a continuous, stable internal anti-TAM effect, with a consequent more constant and durable tumor control. In this context, the antitumoral effect of radiotherapy is enhanced by persistency of microparticles in the tumor tissue.

Therefore, the invention provides a tumor therapy using a sub-optimal tumor-volume coverage dose of radioactivity combined with microparticles deposition as a mean of tumor control. In this therapy, the direct cytotoxic effect of the ionizing radiations on tumor cells is enhanced by the depletion of pro-tumoral macrophages from the treated tumor induced by local microparticle deposition and the induction of anti-tumor immunity mechanisms through the recruitment of external macrophages and dendritic cells and the activation of T cells lymphocytes production. This approach is able to generate a diffuse antitumoral effect both within the tumor mass and in the surrounding infiltrative area. The combined antitumoral effect of the radioactivity, pro-tumoral macrophage depletion from the treated tumor and anti-tumor immune cells recruitment activation allows the total absorbed dose of radioactivity required for the tumor killing effect to be reduced as compared to standard external beam radiotherapy (EBRT) treatment, with a direct consequence of sparing the surrounding healthy tissue from radiation-induced toxicity.

The radioactive microparticles for the use according to the invention are made of material(s) which can be loaded or doped with radioactive and non-radioactive isotopes to form biocompatible and biopersistent radioactive microparticles. For example, the isotopes may be loaded onto the microparticles through impregnation, absorbing or covalent binding, either directly or through appropriate linker or chelator, using standard techniques that are well-known in the art.

The radioactive microparticles for the use according to the invention have a good penetration into the tissue and good fixation within the tumor after injection, without migration out of the tumor. In particular, the radioactive microparticles for the use according to the invention are biopersistent and remained at the injection site within the tumor without leaching any radioisotope in the healthy tissues from the treated patient. Therefore, the potential damages to the surrounding tissues and the toxic effects to the treated patient are limited with the radioactive microparticles according to the invention. The radioactive microparticles for the use according to the invention are capable of inducing the depletion of pro-tumoral macrophages from the treated tumor after their local deposition inside the tumor. The pro-tumoral macrophage depletion effect is also present in the non-radioactive microparticles (non-activated microparticles comprising the non-radioactive isotope). The radioactive microparticles for the use according to the invention have also a very good colloidal stability favorable to injectability and are not altered by neutron activation. These properties depend on the intrinsic characteristics of the microparticles suspensions such as in particular, concentration, stability, particle size distribution, and/or viscosity. These characteristics can be easily determined using standard method that are well-known in the art and adapted by those skilled in the art to obtain radioactive microparticles having the desired properties.

Non-limiting examples of microparticles for use according to the invention include: acetlyacetonate (AcAc), resin, ceramics including glass, plastics, natural or synthetic polymers, other materials and combinations thereof. Examples of polymers for use in the present invention include with no limitations: polysiloxane, Poly L-Lactic Acid (PLLA), alginate, chitosan and combinations thereof. Polysiloxane particles are disclosed for example in Patent FR 2 930 890 and Marcon et al., J. Nanomed. Nanotechnol., 2017, 8, 5; DOI: 10.4172/2157-7439.1000460). PLLA particles are disclosed for example in Zielhuis et al., Int. J. Pharm., 2006, 311, 69-74; Bult et al., Pharm. Res., 2012, 29, 827-836. Chitosan particles are described for example in US 2008/0166297. In some embodiments, the microparticles comprise or consist of polysiloxane, acetlyacetonate (AcAc), Poly L-Lactic Acid (PLLA), alginate, chitosan, resin, glass or combination thereof. In some preferred embodiments, the microparticles comprise or consist of polysiloxane.

The particle size distribution (granulometry) is an important parameter of the microparticles. Indeed, particle size is closely related to the distribution of the microparticles in vivo, but also to their elimination. The microparticles usually have a mean or median size from 0.1 μm to 60 μm. In some embodiments, the microparticles have a mean or median size from 0.1 μm to 10 μm, preferably 0.1 μm to 1 μm, more preferably 0.3 μm to 0.6 μm, even more preferably 0.4 μm to 0.5 μm. In some preferred embodiments, the microparticles are microspheres having a mean diameter from 0.1 μm to 60 μm, preferably 0.1 μm to 10 μm or 0.1 μm to 1 μm, more preferably 0.3 μm to 0.6 μm, even more preferably 0.4 μm to 0.5 μm.

In some embodiments, the radioactive isotope is a beta-negative or alpha emitting radioisotope or a combination thereof, preferably a short half-life and high energy beta-negative emitting radioisotope or a combination thereof. Examples of radioisotopes that can be used in the invention include: holmium-166 (Ho-166 or ¹⁶⁶Ho), lutetium-177 (Lu-177 or ¹⁷⁷Lu), rhenium-186 or -188 (Re-186 (¹⁸⁶Re) or Re-188(¹⁸⁸Re)), gold-198 (Au-198 or ¹⁹⁸Au), yttrium-90 (Y-90 or ⁹⁰Y), thorium-227 (Th-227 or ²²⁷Th), actinium-225 (Ac-225 or ²²⁵Ac) and combinations thereof.

| | | | | α |
|---|---|---|---|---|
| | Half-life | β⁻ energy | γ energy | energy |
| Radioisotope | (days) | (keV) | (keV) | (keV) |
| Holmium-166 | 1.1 | 1840 | 80 | No |
| Lutetium-177 | 6.7 | 497 | 208 | No |
| Rhenium-186 | 3.8 | 1077 | 137 | No |
| Rhenium-188 | 0.71 | 2100 | 155 | No |
| Yttrium-90 | 2.7 | 2080 | No | No |
| Gold-198 | 2.7 | 1372.9 | 411.8 | No |
| Thorium-227 | 18.7 | No | 235.9 | 6146 |
| Actinium-225 | 10 | No | 218 (Fr-211 | 5935 |
| | | | daughter) | |
| | | | Or 440 (Bi-213 | |
| | | | daughter) | |

In some preferred embodiments, the radioisotope is holmium-166. Compared to other radioisotopes, Ho-166 is of particular interest for the use of the present invention as it represents a very good compromise, combining a short half-life and high β⁻ energy. In addition, Ho-166 is both a β⁻-emitter, and a γ emitter, so coupling a high therapeutic efficacy to the possibility of detection by Single photon emission computed tomography (SPECT) enabling nuclear imaging, relevant for personalized patient treatments, in particular for dosimetric calculations. Compared to other β⁻ and γ emitters used in medicine (Lu-177; Re-186 and Re-188), Ho-166 has a β⁻ energy and relative penetration range in tissue (maximum 8 mm; about 6 mm) which are more suitable for application in local radiotherapy (brachytherapy) than lutetium (Lu-177). Due to the very limited tissue penetration of its β-particles, Ho-166 allows a much higher local radiation dose compared to external beam radiotherapy, EBRT, (typically 200 up to >1000 Gy in a close vicinity of the deposition site possibly reaching up to ten of thousands of cells.

In some embodiments, the microparticles comprise a radioactive and a non-radioactive isotope from the same chemical element such as holmium-165 and holmium-166. The radioactive isotope is advantageously produced by standard neutron activation techniques that are well-known in the art, such as for example using a neutron activator to allow the activation of a fraction of the microparticles through neutron capture. Neutron activation is disclosed for example in the patent FR 2 930 890. The microparticles are stable during neutron activation.

In some preferred embodiments, the microparticles such as polysiloxane microparticles, comprise holmium-165 and holmium-166. Preferably, the microparticles such as polysiloxane microparticles, comprise at least 20% (w/w) of total holmium, more preferably at least 25% (w/w) of total holmium. Total holmium content refers to total holmium content, which means holmium-165 and holmium-166. Holmium content is determined by standard analytical methods such as for example Inductively Coupled Plasma Mass spectrometry (ICP-MS). A high holmium content will potentially enable the delivery of a high radioactive dose to tumor cells.

Ho-165, the stable natural isotope from which Ho-166 is obtained by neutron activation is monoisotopic and therefore easier to control for both activation and imaging/dosimetry compared to natural rhenium which comprises two isotopes, yielding two radioisotopes by neutron activation, Re-186 and Re-188, with different characteristics. Furthermore, Ho-165 remains predominant within microparticles after irradiation. Moreover, holmium is highly paramagnetic and has a high mass attenuation (X-ray absorption) coefficient making this atom detectable by Magnetic Resonance Imaging (MRI), ultrasound and Computed tomography (CT) techniques. Multimodal imaging is therefore possible with Ho-165/Ho-166 microparticles, paving the way to determine the optimum distribution of microparticles once injected, and also for dosimetry estimations. Therefore, Ho-165/Ho-166 microparticles such as Ho-165/Ho-166 polysiloxane microparticles, combine advantageously multimodal imaging and therapeutic efficacy.

In some embodiments, the microparticles, preferably Ho-165/Ho-166 microparticles such as Ho-165/Ho-166 polysiloxane microparticles, have a specific activity of at least 1 megabecquerel (MBq) per milligram, for example 1 to 10 MBq per milligram, such as in particular 2 to 3 MBq per milligram.

The composition for the use according to the invention is a pharmaceutical composition comprising a therapeutically effective amount of microparticles, preferably Ho-165/Ho-166 microparticles, such as Ho-165/Ho-166 polysiloxane microparticles, suspended in a pharmaceutically acceptable injection vehicle. The microparticles are suspended in the injection vehicle to form a stable and injectable suspension.

The amount or concentration of microparticles in the composition for use according to the invention is reflected by its dry matter (w/v) content or its density (w/v). High values of density or dry matter refer to a composition that is highly concentrated. The dry matter is linked to the concentration of microparticles obtained after synthesis and dispersion in aqueous solution. The concentration of particles is directly related to the specific activity produced during the activation, and therefore to the therapeutic activity induced after injection. These parameters can be easily determined using standard method that are well-known in the art and adapted by those skilled in the art to obtain a pharmaceutical composition having the desired therapeutic activity.

The concentration of microparticles, sedimentation rate and viscosity of the pharmaceutical composition are adapted to formulate a stable and injectable suspension. The sedimentation rate informs about the colloidal stability, for a proper injection of the suspension. The viscosity is an important parameter to evaluate the correct circulation of the suspension flow in the fluidic line of the injector system during the intratumoral injection.

In the context of the invention, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose treatment procedure is determined and adjusted depending on factors such as the composition used, the route of administration, the physical characteristics of the individual under consideration such as sex, age and weight, concurrent medication, and other factors, that those skilled in the medical arts will recognize.

The treatment dose is adjusted to the solid tumors characteristics, such as shape, volume size and location within the body, especially when located to “organ at risks”.

A “pharmaceutically acceptable vehicle” refers to a vehicle that does not produce an adverse, allergic or other untoward reaction when administered to a mammal, especially a human, as appropriate.

The pharmaceutical composition contains vehicles, which are pharmaceutically acceptable for a formulation capable of being injected. These may be a fluid carrier, in particular an aqueous carrier such as sterile water, isotonic, sterile, saline solutions (monosodium or disodium phosphate, sodium, potassium, calcium or magnesium chloride and the like or mixtures of such salts), dextrose solution, mannitol such as Ringer's solution, Ringer-lactate, Hank's solution and other physiologically balanced salt solution. The pharmaceutical forms suitable for injectable use include in particular sterile aqueous suspensions. The suspension may comprise additives which are compatible with radioactive particles such as substances that enhance isotonicity and chemical stability. In all cases, the form must be sterile and must be fluid to the extent that easy syringe ability exists. It must be stable under the conditions of manufacture and storage and must be preserved against the contaminating action of microorganisms, such as bacteria and fungi.

The pharmaceutical composition of the invention has a pH and osmolality that are in agreement with those tolerated by the treated patient. For example, the pH may be from about 6 to about 9 and the osmolality from about 100 mOsm to about 300 mOsm.

In some embodiments, the composition comprises an aqueous suspension of the radioactive microparticles, preferably Ho-165/Ho-166 microparticle suspension, such as Ho-165/Ho-166 polysiloxane microparticle suspension, in an injection vehicle comprising water and eventually low amount of ethanol, preferably less than 5% (v/v).

In some embodiments, the microparticle suspension, preferably Ho-165/Ho-166 microparticle suspension, such as Ho-165/Ho-166 polysiloxane microparticle suspension, has a density of at least 1 g/mL, for example 1 g/mL to 1.5 g/mL. Alternatively or additionally, the microparticle suspension may have a dry matter content from about 400 g/L to about 700 g/L.

In some embodiments, the microparticle suspension, preferably Ho-165/Ho-166 microparticle suspension, has a viscosity of at least 3 mPa·s, preferably from 3 mPa·s to 12 mPa·s, more preferably 6 mPa·s to 10 mPa·s. Alternatively or additionally, the microparticle suspension may have a colloidal stability characterized by less than 15% of sedimentation after 1 h or less than 25% sedimentation after 24 h, as determined by the method of Westergren described in Miller et al., Br. Med. J. (Clin. Res. Ed.), 1983, 286:266-.

The microparticle suspension is stable in vitro and in vivo. In particular, the microparticle suspension is stable in vitro during the intratumoral administration phase to the patient, for several hours. The microparticle suspension is stable in vivo and persists at the injection site inside the tumor for several months, as shown in the examples of the application.

The pharmaceutical composition of the present invention is administered to the patient by intratumoral administration (injection directly into a tumor) according to known procedures, at dosages and for periods of time effective to induce a therapeutic effect in the patient. The composition may be administered through single or multiple injection locations of an appropriate volume of injection, at an appropriate injection rate, using suitable injection devices that are well-known in the art. For the safety and efficacy of the treatment, the intratumoral administration of the radioactive microparticle suspension is performed in a controlled manner and monitored. For example, stereotactic injection may be combined with image-guided administration using Computerized Tomography (CT) or ultrasound. Average radiation dose adsorbed in tumor (Gray) is calculated from tumor imaging data using appropriate softwares that are well-known in the art such as Monte Carlo simulation software Geant4 Application for Tomographic Emission (GATE) and the like.

In some embodiments, the invention comprises the administration of a sub-optimal tumor-volume coverage dose of radioactivity. In some preferred embodiments, the invention comprises the intratumoral administration of an absorbed dose superior or equal to 100 Grays in less than 95%, 85%, 75%, 65%, 55%, 45% or 35% of the tumor volume, preferably in 35% to 55% of the tumor volume. In other preferred embodiments, the invention comprises the intratumoral administration of an absorbed dose superior or equal to 60 Grays in less than 95%, 85%, 75%, 65%, 55% or 35% of the tumor volume, preferably in 35% to 55% of the tumor volume.

As shown in the examples of the present application a sub-optimal tumor-volume coverage dose of radioactivity delivered by the composition according to the invention is sufficient to obtain a therapeutic effect in the treated cancer patient. The therapeutic effect against cancer is characterized by a total remission or a stable disease. As used herein, the term “total remission” refers to the elimination of the tumor and the absence of clinical signs in the treated cancer patient. As used herein, the term stable disease, refers to the reduction of the tumor mass that is stable over time with limited or no clinical signs in the treated cancer patient.

The entire tumor will be treated by the combined antitumoral effect of direct killing by ionizing radiation absorbed dose, elimination of pro-tumoral macrophages (TAMs) and activation of anti-tumor immunity mechanisms (recruitment of external macrophages, dendritic cells and production of T cells lymphocytes). Since, the method invention allows the use of sub-optimal tumor-volume coverage dose of radioactivity, the peripheral area of the tumor will receive a dose of radioactivity inferior to the estimated minimum absorbed dose (60Gy to 100 Gy) to prevent the damage to the healthy tissues surrounding the tumor.

In some embodiments of the invention, the cancer treatment comprises a single intratumoral administration of the composition or multiple intratumoral administrations over time. As shown in the examples of the present application a single administration of sub-optimal tumor-volume coverage dose of radioactivity delivered by the composition according to the invention is sufficient to obtain a therapeutic effect in the treated cancer patient.

In some embodiments, the intratumoral administration of the composition is used in combination with one or more other anticancer treatments such as surgery, external radiotherapy, chemotherapy, and immunotherapy, wherein the treatments are administered simultaneously, separately, and/or sequentially.

The treatment of cancer according to the invention comprises one or more of: inhibiting the growth of a solid tumor, inhibiting proliferation of solid tumor cells, inhibiting solid tumor metastases, reducing tumorigenicity of solid tumor cells, reducing the frequency of cancer stem cells or tumor initiating cells in a solid tumor, and generating a diffuse antitumoral effect both within the tumor mass and in the surrounding infiltrative area.

The solid tumors treated using radioactive microparticles according to the present invention may be defined by grade, and may be low grade, medium grade, or high grade solid tumors.

In some embodiments, the solid cancer is a tumor with a macrophage component, such as with no limitations: glioblastoma, glioma, Kaposi's sarcoma, breast, ovarian, lymphoma, colon, stomach, lung or prostate cancer and others.

In some embodiments, the solid cancer is a solid tumor that cannot be resected or is not responsive to current radiotherapies or chemotherapies, such as with no limitations: glioblastoma, glioma, Kaposi's sarcoma and others.

In some embodiments, the cancer is selected from the group comprising: bladder, brain, breast, colon, gastric, eye, head and neck, skin, kidney, liver, lung, ovary, oral, prostate, small intestine, soft-tissue, testis, thyroid, and uterus cancer. Preferably, the cancer is brain cancer.

Brain cancer includes primary brain tumors, metastatic brain tumors and skull base cancers. Brain cancer includes low to high grade gliomas such as astrocytoma, oligodendroglioma, ependydoma and glioblastoma; meningioma; primitive neuroectodermal tumors; pituitary tumors; pineal tumors; choroid plexus tumors; teratoma; neurocytoma; dysembroplastic neuroepithelial tumor; lipoma; hemangioblastoma; hamartoma; neuroma; shwannoma; neurofibroma; metastatic brain tumors such as in particular brain metastatic adenocarcinoma; carcinomatous meningitis; and mixed brain tumors. Skull base cancer includes chondroma, chordoma, sarcomas, gliosarcoma, chondrosarcoma and rhabdomyodarcoma. In some preferred embodiment, the brain cancer is glioblastoma or metastatic brain tumors such as brain metastatic adenocarcinoma.

One aspect of the invention relates to a method of treating cancer in a patient in need thereof comprising: administering to a patient a therapeutically effective amount of the pharmaceutical composition as described above.

In the above embodiments, the microparticles are preferably Ho-165/Ho-166 microparticles, in particular Ho-165/Ho-166 polysiloxane microparticles; preferably comprising at least 20% (w/w) of total holmium, more preferably at least 25% (w/w) of total holmium; and/or preferably having a mean or median size from 0.1 μm to 1 μm, more preferably 0.3 μm to 0.6 μm, even more preferably 0.4 μm to 0.5 μm; and/or preferably having a specific activity of at least 1 megabecquerel (MBq) per milligram, for example 1 to 10 MBq per milligram, such as in particular 2 to 3 MBq per milligram. Preferably, the composition comprises a microparticle suspension having a density of at least 1 g/mL, for example 1 g/mL to 1.5 g/mL; and/or a viscosity of at least 3 mPa·s, preferably from 3 mPa·s to 12 mPa·s, more preferably 6 mPa·s to 10 mPa·s. Preferably, the composition comprises an aqueous suspension of microparticles in an injection vehicle comprising water and eventually low amount of ethanol, preferably less than 5% (v/v).

In the above embodiments, the microparticles, preferably Ho-165/Ho-166 microparticles, in particular Ho-165/Ho-166 polysiloxane microparticles such as defined above are preferably used for the treatment of brain cancer as defined above, in particular glioblastoma.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### Figure Legends

FIG. 1 represents the Kaplan-Meier survival curve of group 1 (Ho-166/Ho-165), group 2 (Ho-165) and the control group. This graph demonstrates that the survival time for group 1 was significantly longer than the two other groups.

FIG. 2 is a representative example of the images acquired from D14 onwards (after injection of contrast agent).

A. Tumor size 1.5 cm just before Holmium (Ho-166/Ho-165) microparticle suspension injection,

B. Presence of holmium-166 just after intratumoral injection of Holmium (Ho-166/Ho-165) microparticle suspension at D14 in the center of the glioblastoma (GB),

C. Tumor condition 15 days after intratumoral injection of Holmium (Ho-166/Ho-165) microparticle suspension. Tumor size is regressing,

D. Tumor at 25 days after intratumoral injection of Holmium (Ho-166/Ho-165) microparticle suspension. Tumor size is still regressing,

E. Tumor condition 65 days post-injection of Holmium (Ho-166/Ho-165) microparticle suspension, just before animal euthanasia at the end of observation period (2 months post-injection of holmium). A small volume of tumor is observable,

F. Tumor condition 65 days after intratumoral injection of Holmium (Ho-166/Ho-165) microparticle suspension, just before animal euthanasia at the end of the observation period. This image was acquired from the minipig in which the tumor was no more observable from 45 days post-injection of Holmium (Ho-166/Ho-165) microparticle suspension, suggesting a complete response

G. CT-scan of the animal (group 1) in which the Holmium (Ho-166/Ho-165) microparticle suspension was injected outside the tumor. Tumor growth continued (arrow) on the opposite side from the holmium-166 injection area,

H. CT-Scan of a minipig from group 2 (holmium-165 microparticle suspension) acquired before euthanasia. Tumor volume increased,

I. CT-Scan of a minipig from the control group acquired before euthanasia. Tumor volume increased.

### Example 1: Holmium-165 (165Ho) Siloxane Particles Synthesis

Nanostructured Ho2O3 precursor (0.397 mol) was slurried and refluxed in 1.5 L ethanol together with acetic acid (0.262 mol) and Si-EDTA (0.05 mol). It was allowed to cool down to ambient temperature and then transferred into 200 mL plastic centrifuge bottles. Particles were washed in ethanol twice by centrifugation cycles of 10 minutes at 4,100 rpm and then resuspended in 160 mL mQ water. Particle size was homogenized by stirring the suspension for 4 days at 30° C. The objective was to synthesize particles with high holmium-165 content. In this study, the particle mean size was 470 nm. The dry matter concentration (i.e. the solid phase once completely dried) was 550±50 g/L consisting of 28±3% holmium weight content. This elevated fractional weight of holmium enables the delivery of a highly activated holmium-166 dose to tumor cells within a low and suitable volume unit of treatment. Density of the final holmium-165 microparticle suspension was 1.38 g/ml, measured in triplicate at 25° C. by weighing 1 ml of suspension.

### Example 2: Activation of holmium-166 Microparticles Suspension

A volume of 1 ml of the holmium-165 suspension aimed for one tumor was withdrawn from the initial vial and filled in a thermoplastic PolyEtherEtherKetone (PEEK) capsule suitable for the activation process. Each capsule was weighed before and after the activation process. Then the holmium-166 was produced within the suspension of microparticles with a neutron activator (Advanced Accelerator Applications) coupled to a 70 MeV cyclotron (IBA, C70 of GIP Arronax facility). The principle of the activation consisted of the production of a neutron flux generated by the interaction of a proton beam (70 MeV) and a metallic target. The neutrons then interacted with the suspension of microparticles and through neutron capture a part of the stable atoms of holmium (holmium-165) were transformed into activated holmium (holmium-166). In nuclear physics, this neutron capture reaction is written as holmium-165 (n,γ) holmium-166 since when capturing an incident neutron, the nucleus also emits a gamma ray to compensate the excessive energy provided by the neutron, returning to his fundamental energy state. The samples were produced in order to obtain specific activities at the time of injection of 2 to 3 MBq/mg of microparticles.

### Example 3: Direct Cytotoxic Effect of the Cold Microparticles on the Monocyte/Macrophages

In order to assess possible effects of cold microparticles per se on potential activation of immune system, Ho-165 microparticles (prepared as described in Example 1) were tested undiluted and diluted at the following concentrations (1:10, 1:100, 1:1000) to evaluate immunotoxicity effects in healthy volunteer whole heparinized blood. Immunophenotyping analysis by FACS was performed after incubation with whole blood. CD3 (T-cell marker), CD4 (T-helper cell marker), CD8 (cytotoxic T-cell marker), CD14 (Monocyte differentiation antigen), CD19 (B lymphocyte antigen), CD45 (Leukocyte Common Antigen, LCA) and CD69 (Very Early Activation Antigen, VEA) cell surface marker were evaluated. Ho-165 microparticles diluted 1:10, 1:100 and 1:1000 were incubated in whole blood at 37° C. for 4 h. PMN and lymphocytes were not affected, while a reduction of Monocyte after incubation with Ho-165 microparticles diluted 1:10 was observed. Leukocyte formula was also investigated. Both T helper (CD3+CD4+), cytotoxic T cells (CD3+CD8+) as well as the activated subpopulation (CD4+CD69+ and CD8+CD69+) was not affected by treatment with Ho-165 microparticles (1:10 and 1:100). Treatment of whole blood with Ho-165 microparticles 1:10 and 1:100 did not affect normal (CD3-CD19+) and activated B cells (CD3-CD19+ CD69+), whereas dilution 1:10 was able to reduce the percentage of monocyte. These results show that cold microparticles per se have a direct effect on monocyte/macrophages.

### Example 4: The Cold Particles Activate the Complement Pathway

Cold Ho-165 microparticles (prepared as described into Example 1) were tested for evaluation of blood interactions investigating possible alteration of coagulation, platelet activation and complement system activation. For coagulation investigation, WB was incubated for 4 h at 37° C. with Ho-165 microparticles diluted 1:10, 1:100 and 1:1000. Ho-165 microparticles did not induce any change in Thrombin-Antithrombin Complex (TAT) levels quantified by ELISA method. Effects of Ho-particles on platelet activation was evaluated by measuring surface expression of two activation markers (CD62P and AnnexinV) after 30 minutes of incubation at 37° C. of platelet-rich plasma obtained by centrifugation of WB and Ho-165 microparticles (1:10, 1:100 and 1:1000). No modification of platelet activation was recorded after incubation with Ho-165 microparticles. Influence of complement system activation was evaluated after 1 h of incubation at 37° C. of human sera obtained from WB and Ho-165 microparticles (1:10, 1:100 and 1:1000). Ho microparticles at the highest concentration tested (1:10) induced an activation of the complement pathway.

These in vitro data on monocytes suggest that the cold Ho-165 microparticles have an antitumoral effect through the suppression of TAMs and/or activation of anti-tumoral macrophages.

### Example 5: Persistence of the Microparticles at the Injection Site

The tolerability to Holmium particles (formulated as described into Example 1) was investigated in Sprague Dawley rats, after a single intramuscular administration of 55 mg of microparticles followed by observation periods of 4 and 12 weeks, in order to evaluate the persistency of Holmium particles in situ and to perform a preliminary toxicity evaluation of Holmium particles in rats. Daily clinical signs, body weight, hematology and clinical chemistry investigations, macroscopic observations and organ weights were performed on all animals. Histopathological evaluation of the injection site was performed as well. No mortality occurred and no signs of systemic toxicity were seen in animals sacrificed after 4 or 12 weeks of treatment-free period. On the other hand, there were obvious signs that indicated granulomatous reaction, associated with fibrosis, indicating that the injected particles remained in the administration site. On the basis of the results obtained in this study, it can be concluded that the Holmium particles, at the dose tested, did not induce any systemic toxicity, while a low local tolerability was observed.

The aim of the present invention is to use activated/cold microparticles for treatment of solid tumors such as glioblastoma. It is known from the literature that radiation could influence the inflammatory response. In particular, low-dose irradiation predominantly induced anti-inflammatory activation of macrophages while high dose irradiation was more prone to enhance the pro-inflammatory properties of macrophages. The combination of the effect of cold and radioactive particles can be used in the treatment of tumors with a strong macrophage microenvironment and was investigated in an in vivo model of glioblastoma in pigs.

### Example 6: Therapeutic Efficacy of Ho-166/Ho-165 Microparticle Suspension on Induced Glioblastoma in Minipig Tumor Model

The glioblastoma (GB) infiltration is a great problem (Houben et al., Ned. Tijdschr Geneeskd. 2005, 149, 2268-72) that makes the complete treatment of this tumor impossible with current therapeutic methods (Urbańska et al., Termedia Publishing, 2014, 18, 307-12). In this study, the therapeutic efficacy of holmium-166 (Ho-166/Ho-165) microparticle suspension was evaluated after its intratumoral injection in a GB minipig model. This radioactive source was selected due to its reasonable half-life (26.8 h), and its ability to emit both low-energy gamma rays (used for imaging) and high-energy beta particles (suitable for tumor treatment). Beta particles have a restricted infiltration in tissue resulting in a minimal irradiation of the healthy tissue (Schwartzbaum et al., Nat. Clin. Pract. Neurol., 2006, 2, 494-503).

Ho-166/Ho-165 microparticle suspension was administered intratumorally, in a group of minipigs, two weeks after implantation of tumor cells (U87 glioblastoma cell line) (D14) using a stereotactic frame coupled with a preclinical injection system. The results were compared with two other groups of minipigs: one group treated with the non-activated microparticle suspension (holmium-165), and one group without any treatment (control group), under the same operating procedure. The criteria of treatment efficacy were the regression (repression) of the tumor growth and the overall survival time after the intratumoral injection of holmium-166 microparticles.

### Materials and Methods

U87 Cells Culture

U87 cells, a human primary glioblastoma (GB) cell line formally known as U-87 MG (Uppsala 87 Malignant Glioma) were chosen because U87 is one of the most radioresistant (Hovinga et al., J. Neurooncol. 2005, 74, 99-103; Naidu et al., J. Radiat. Res. 2010, 51, 393-404). They were prepared as previously reported to induce GB on landrace pigs (Selek et al., J. Neurosci. Methods., 2014, 221, 159-165) and Yucatan minipigs (Khoshnevis et al., J. Neurosc.i Methods. Elsevier, 2017, 282, 61-68). Briefly, 35-50×10⁶ U87 cells were harvested from two T175 tissue culture flasks after one week in growing medium (Minimum Essential Media (MEM) supplemented with 10% fetal calf serum, 100 U/ml penicillin, 100 μg/ml streptomycin, 2 mM I-Glutamine and 0.25% g/mL amphotericin B) with three medium changes. Cells were processed with 0.25% trypsin-EDTA to be harvested and then washed twice in PBS before being pelleted for 4 min at 3000 g in a 2 ml Eppendorf vial just before the inoculations. The final cell concentration in each Eppendorf vial was about 1.7×10⁶ cells/10 μl. For each subject, 40 μl of the cells were injected into the right hemisphere.

Animals

The experiments were performed using 12 Yucatan minipigs as a large animal glioblastoma (GB) model. The minipigs were aged between 3-4 months old and both sexes were used. The minipigs were housed under conventional conditions (temperature=19° C., humidity >35% and ventilation 10 times/hour) with ad libitum access to tap water and given pelleted feed twice a day. A quarantine period of 2 weeks was allowed as acclimatization period before the implantation of U87 cells.

Holmium-165 Microparticles Preparation

Suspensions composed of holmium-165 (stable isotope) microparticles were synthesized according to the experimental procedure described in example 1.

Activation of Holmium-166 Microparticles Suspension

Then the holmium-166 was produced within the suspension of microparticles with a neutron activator according to the experimental procedure described in example 2.

Stereotactic Frame

A large animal stereotactic instrument, obtained from RWD Life Science Inc. (Model 68901) was selected for the intracerebral implantation of the U87 cells and then the intratumoral injections of radioactive holmium. Before any injections, the location of the tumor is obtained with a pre-operative CT-Scan.

Treatment Planning

The treatment planning (TP) covered different % (35, 45, 50, 60, 80 and 97%) of the tumor volume (TV) with at least 100 Gy (considered as the tumor killing dose). In all injected minipigs, the TP was performed based on the pre-operative CT-scan acquired, at day 14 post-implantation (D14), 30 minutes before the injection of holmium suspension. Performing the pre CT-scan as late as possible meant that the planning was performed with the most up-to-date information regarding the tumor's size, shape and position. For the CT-acquisition, product contrast was used so as to easily differentiate the tumor from healthy tissue. Tumor segmentation was then performed with the open-source software, 3D Slicer. Once segmentation had been completed, TPS was performed with the optimization algorithm, the non-dominated sorting genetic algorithm II (Srinivas et al., Evol. Comput. MIT Press; 1994, 2, 221-248). The volume of each injection's unit of treatment (UoT) varied between 5 and 8 μl, depending on the tumor size and as a result of the calculation aiming to decrease the number of injections and reduce the duration of surgery while delivering the targeted absorbed dose and volume coverage (Table 1).

Immunosuppressive Therapy

Immunosuppression of the animals was used to prevent grafted cell rejection throughout the duration of experiment. This works by weakening the immune system to accept the implanted cells as if they were their own. Cyclosporine solution (Neoral® 100 mg/ml) was administered orally, twice a day (25 mg/kg). The cyclosporine treatment continued each day until the last day of the experiment.

Tumor Cell Transplantation and Intratumoral Injection of Holmium Microparticles

12 Yucatan minipigs were anesthetized and the tumor cells were injected into the brain in the corpus striatum area in the right hemisphere at D0. Fourteen days after tumor cell transplantation, the minipigs were divided into two groups, with group 1 receiving injections of Ho-166/Ho-165 suspension and group 2 receiving injections of Ho-165 suspension (non-radioactive). In the control group, no injections were performed after tumor cell implantation. For the three different groups, six minipigs were in group 1, three were in group 2 and 3 were in the control group. All the suspension preparation and intratumoral injection procedures were identical between the radioactive and non-radioactive holmium injections, with the radioactive injections respecting required radioprotection protocol. One pig of Group 1 (Positive fail pig) was excluded from group 1 after an error of needle positioning that has led to the injection of the radioactive suspension outside t of the tumor. According to the tumor position as determined by the pre-operative CT images acquired just before the holmium injection, the needle was inserted into the brain. Several Unity of Treatment were injected inside the tumor in different sites using the coordinates given by the TP. The number of injections varied between tumors and the % of the tumor needing to be treated with a dose >100Gy.

CT-Scan Imaging

To study the distribution of holmium inside the tumor and evaluate the development of tumor size over time, all minipigs were periodically scanned via CT-Scan. A CT-Scanner (GE BrightSpeed 16) to perform all CT acquisitions. After cell implantation, tumor growth was assessed in all minipigs with imaging on days 7, 10 and 14. In the 9 minipigs that underwent the treatment (excluding the 3 in the control group), pre- and post-operative CT imaging were performed on the day of holmium suspension injection (D14) to assess tumor positioning and to verify the distribution and location of injected holmium, respectively. After the treatment, the evolution of injected holmium suspension and the therapeutic effect of this radiation therapy were studied by performing the first post-injection CT-Scan, 5 days after the intervention and then every ten days following that. Follow-up CT acquisitions for the minipigs in the control group were the same as those performed for groups 1 and 2. In all minipigs, a manual segmentation of the tumor was performed after each post-operative CT-acquisition. In this fashion, the changes of tumor size could be studied over time.

Follow-Up

A standard follow-up period was used for the 2 months following treatment. In this period, all the animals were monitored for clinical side effects on a daily basis. Clinical parameters observed consisted of food intake, body temperature, weight, alertness, consciousness levels, posture and gait.

Histology and Immunochemistry

Histological and immunohistochemical analyses were performed. Samples from brain tumor, heart, liver, lung and kidney were collected, fixed in 4% formalin, routinely processed and embedded in paraffin. For each sample one 4 μm section was obtained and stained with hematoxylin and eosin (HE). For each brain sample, 7 sections were cut and used for immunohistochemistry. Immunohistochemical stains were performed with the avidin-biotin-peroxidase complex method. Lymphoid infiltrate was characterized by using anti-CD3 epsilon (diluted 1:100, Bio Rad, Hercules, Calif., USA) for T lymphocytes and anti-Pax-5 (clone 24/Pax5, undiluted, BD Transduction laboratories, Lexington, Ky., USA) for B lymphocytes. In order to detect the macrophage/dendritic cell (DC) population, the following antibodies were applied: anti-Iba-1, (ab5076, diluted 1:500, Abcam, Tokyo, Japan), which is a panmarker for macrophages/DCs, anti-CD204 (MSR-1, clone SRA-E5, diluted 1:50, Abnova, Taipei, Taiwan), which labels conventional macrophages/monocytes but not DCs, anti-DC-lamp (CD208, clone 1010E1,01, diluted 1:200, Dendritics, Lyon, France), which labels conventional antigen-presenting DCs but not macrophages and anti-CD303 (BRDA-2, clone 124B3, diluted 1:200, Dendritics, Lyon, France), which labels plasmacytoid DCs. For all the antibodies applied, except for anti-DC-lamp, antigen retrieval was performed by heating at 90° C. for 40 minutes in citrate tampon pH6, followed by a cool off. Antigen retrieval for anti-DC-lamp was performed in a pH9 solution, (Dako target retrieval solution, pH9, Dako, Carpinteria, Calif.) at 90° C. for 40 minutes. Labeling was amplified with the last step-product of the ultraTek HRP (anti-polyvalent) Ready to use kit (ScyTek) (30 minutes at 20° C.) and revealed with Vector NovaRED Peroxidase (HRP) Substrate kit (5 minutes). Hematoxylin counterstain (10 seconds) was applied; then sections were dehydrated and mounted.

For each antibody a negative control, characterized by the replacement of the first antibody with PBS solution, and a positive control for each antibody applied, represented by a 4 μm section of spleen from a control group pig, were used.

Scoring and Pattern Identification at Light Microscopy

Histological analysis of the brain was used to confirm the findings of CT images and to observe tumor size and necrosis after the treatment. For postmortem examination, a histological analysis was performed, during which the tumor-bearing section of the brain, the kidneys, the heart, the lungs and the liver were quickly collected and fixed in 4% formalin. The tumor and a small section of the other organs were processed and embedded in paraffin. One 4 μm section was stained with hematoxylin and eosin and histologically examined. On the HE stained section of the brain, the following parameters were scored: presence/absence of tumor, presence/absence of Ho crystals, tumor necrosis, lymphocytic inflammation and granulomatous inflammation. For tumor necrosis, the following semi quantitative score was applied: (−)=absent, (+)=1-30% of the tumor area, (++)=31-60% of the tumor area, (+++)=61-100% of the tumor area. Tumor lymphocytic and tumor granulomatous inflammations were evaluated semi-quantitatively: (−)=absent, (+)=mild, (++)=moderate, (+++)=severe.

Lymphocytic inflammation was then characterized by using anti CD3-epsilon, which labels T lymphocytes, and anti-Pax-5, for B lymphocytes. Plasmacytoid DCs presence was recorded on the basis of anti-CD303 labeling within the tumor. Macrophage/DC infiltrate was evaluated on the basis of positive labeling for anti-Iba-1, anti-CD204, and anti-DC-lamp in different locations: around (peritumoral) and inside (intra-tumoral) the tumor, around the coagulative necrosis and around the Ho associated necrosis, and within the lymphocytic infiltrate. To detect semi quantitatively the amount of positive cells for each antibody and in each location, the following score was applied: (−)=absence of labelled cells, (+)=mild amount of labelled cells, (++)=moderate amount of labelled cells, (+++)=severe amount of labelled cells.

All the histological and immunohistochemical parameters were scored blindly.

Statistical Analysis

Survival data were analyzed using a log-rank test (Mantel-Cox). Statistical analyses were conducted using this nonparametric test which is widely used in preclinical and clinical trials to establish the efficacy of a new treatment in comparison with a control treatment. A value of p<0.01 was taken to indicate statistical significance.

Results

Intratumoral Injection of Holmium and Survival Rate

Intratumoral injections of Ho-166/Ho-165 and Ho-165 microparticle suspensions were performed in groups 1 and 2 respectively. In control group, no treatment and injection was performed after tumor implantation. The number of injections per tumor depended on the tumor size and shape and the % of the tumor treated by >100 Gy (Table 1).

TABLE 1
Number and volume of unit of treatment (UoT), injected in each animal

| | | Number of | Volume of |
|---|---|---|---|
| | | injected UoT | injected UoT |
| Pig | Type of treatment | (μl) | (μl) |
| 1 | Ho-166/Ho-165 | 9 | 5 μl × 3 |
| | | | 6 μl × 6 |
| 2 | Ho-166/Ho-165 | 15 | 5 μl × 3 |
| | | | 6 μl × 4 |
| | | | 8 μl × 8 |
| 3 | Ho-166/Ho-165 | 20 | 5 μl × 8 |
| | | | 6 μl × 12 |
| 4 | Ho-166/Ho-165 | 13 | 5 μl × 13 |
| 5 | Ho-166/Ho-165 | 16 | 5 μl × 13 |
| | | | 6 μl × 3 |
| 6 | Ho-166/Ho-165 | 20 | 5 μl × 11 |
| | | | 6 μl × 9 |
| 7 | Ho-165 | 11 | 5 μl × 9 |
| | | | 6 μl × 2 |
| 8 | Ho-165 | 20 | 5 μl × 16 |
| | | | 6 μl × 4 |
| 9 | Ho-165 | 20 | 5 μl × 3 |
| | | | 6 μl × 17 |
| 10 | Control group | — | — |
| 11 | Control group | — | — |
| 12 | Control group | — | — |

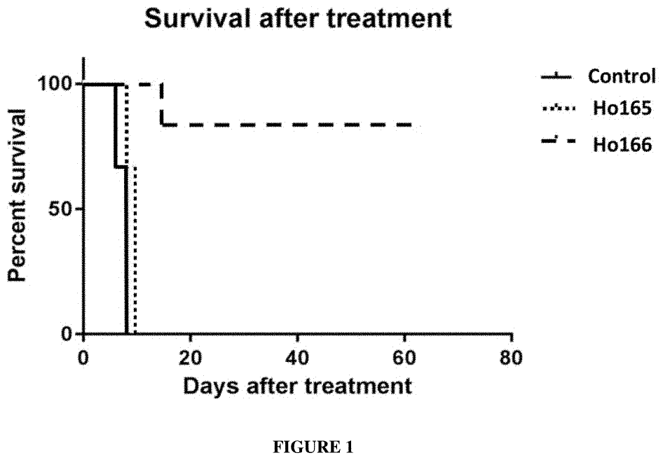

In order to calculate the survival time of the animals in each group, the experimental minipigs were kept alive for 2 months after the treatment date. The Kaplan-Meier survival curve (FIG. 1) was the longest in group 1. For group 2 and the control group, the survival curves were almost the same.

The survival time of all animals and the median survival time of each group were calculated from the date of holmium injection at day 14 post-implantation (D14). The survival time in group 1 was 63 days (Table 2) which is statistically higher (p<0.01) than the survival times in group 2 (8.6 days) and control group (7.3 days) which were not was statistically significant to each other (p=0.09).

TABLE 2
Survival time of all the animals in each group calculated from the day of
holmium injection (D14 post-implantation).

| Group 1 | Group 2 | Control |
|---|---|---|
| (treated with ¹⁶⁶Ho) | (injected with ¹⁶⁵Ho) | group |
| n = 5 minipigs | n = 3 minipigs | n = 3 minipigs |
| 63 | 9 | 8 |
| 63 | 8 | 6 |
| 63 | 9 | 8 |
| 63 | | |
| 63 | | |
| Mean: 63 days | Mean: 8.6 days | Mean: 7.3 days |

In group 2 and the control group, all minipigs demonstrated severe neurologic signs between 6 and 9 days after D14 and consequently they were euthanized. For the “positive fail pig”, untreatable neurological symptoms were observed, which led to the animal being sacrificed 15 days after the treatment.

In group 1, all the pigs (5/5) survived until the end of the observation period, 2 months after D14. At the moment they were euthanized, they were healthy, in a good general condition without any clinical or paraclinical signs of disease.

CT Imaging

During the tumor growth period, several CT-acquisitions were performed, as well as before and after injections of the holmium suspension in groups 1 and 2. At D14, all the implanted tumors demonstrated an increased density in comparison with the surrounding cerebral parenchyma, with clear boundaries and a plurilobed form. According to the post-operative CT-scans in groups 1 and 2, substantial accumulation of microparticles was seen at the injection sites. This indicated that the holmium suspension had been successfully injected intratumorally in all except one minipig in group 1. In this animal, a considerable quantity of Ho-166/Ho-165 suspension was injected outside the lateral part of the tumor.

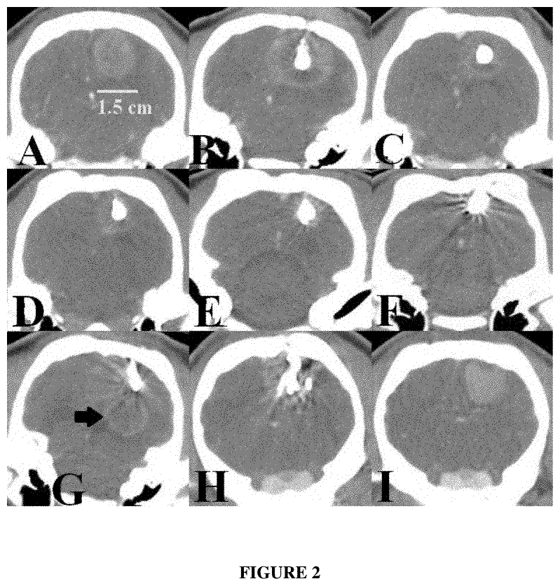

One of the most important points in microbrachytherapy is the retention of the injected suspension in the injection area over time to avoid its leakage outside the tumor. As can be seen in FIG. 2, the Ho-166/Ho-165 suspension was well circumscribed inside the tumor and following scans demonstrating good retention of holmium particles at the site of injection up to 2 months post-injection. This is a very important point for the prevention of damage to normal brain tissue following microbrachytherapy.

A positive response to treatment was obtained in all treated animals in group 1 (Table 3). This means that the Ho-166/Ho-165 microparticles provided good tumor control with no remaining evidence of tumor in the CT acquisitions of three of the animals from 45 days post-injection (FIG. 2). Two other animals showed a significant reduction of tumor size at the end of the 2 months post-treatment observation period. The 5 minipigs treated with Ho-166/Ho-165 microparticles were in good general condition and nutrition state until the day of termination. This confirms the absence of local and systemic toxic effects following an intratumoral administration of holmium-166 suspension. In contrast, tumor growth continued in groups 2 and the control group, leading to animal sacrifice.

TABLE 3
Tumor volumes (mm³) of the 12 minipigs*

| | Pig | Volume tumor (mm³) | | | | | | | | | | |
|---|---|---|---|---|---|---|---|---|---|---|---|---|
| Group | N° | D0 | D7 | D10 | D14 | D19 | D29 | D40 | D50 | D60 | D70 | D80 |
| 1 | 2 | 0 | 263 | 1064 | 2710 | 6787 | 3173 | 1726 | 2045 | 2259 | 2319 | 1596 |
| | 3 | 0 | 279 | 543 | 1789 | 4863 | unobservable | unobservable | unobservable | unobservable | unobservable | unobservable |
| | 5 | 0 | 222 | 460 | 1108 | 2788 | 5724 | 4759 | unobservable | unobservable | unobservable | unobservable |
| | 6 | 0 | 170 | 325 | 1210 | 3099 | 4112 | 1208 | 613 | unobservable | unobservable | unobservable |
| | 4 | 0 | 322 | 239 | 694 | 3229 | 7259 | 2083 | 958 | 905 | 806 | 800 |
| Positive | 1 | 0 | 218 | 419 | 871 | 2843 | Euthanized | Euthanized | Euthanized | Euthanized | Euthanized | Euthanized |
| fail pig | | | | | | | 11124 | at D29 | at D29 | at D29 | at D29 | at D29 |
| 2 | 7 | 0 | 181 | 262 | 579 | 2714 | Euthanized | Euthanized | Euthanized | Euthanized | Euthanized | Euthanized |
| | | | | | | | at D23 | at D23 | at D23 | at D23 | at D23 | at D23 |
| | 9 | 0 | 390 | 572 | 2264 | 8010 | Euthanized | Euthanized | Euthanized | Euthanized | Euthanized | Euthanized |
| | | | | | | | at D22 | at D22 | at D22 | at D22 | at D22 | at D22 |
| | 8 | 0 | 316 | 399 | 1191 | 5237 | Euthanized | Euthanized | Euthanized | Euthanized | Euthanized | Euthanized |
| | | | | | | | at D23 | at D23 | at D23 | at D23 | at D23 | at D23 |
| control | 10 | 0 | 481 | 707 | 787 | 2511 | Euthanized | Euthanized | Euthanized | Euthanized | Euthanized | Euthanized |
| | | | | | | | at D22 | at D22 | at D22 | at D22 | at D22 | at D22 |
| | 11 | 0 | 157 | 342 | 1179 | 5419 | Euthanized | Euthanized | Euthanized | Euthanized | Euthanized | Euthanized |
| | | | | | | | at D20 | at D20 | at D20 | at D20 | at D20 | at D20 |
| | 12 | 0 | 505 | 743 | 928 | 1694 | Euthanized | Euthanized | Euthanized | Euthanized | Euthanized | Euthanized |
| | | | | | | | at D22 | at D22 | at D22 | at D22 | at D22 | at D22 |

*Volumes are based on several CT images acquired after U87 cell implantation. At D14, Ho-166/Ho-165 suspension and Ho-165 suspension were injected intratumorally in groups 1 and 2, respectively.

### Dosimetry

Main observation from dosimetry results (Table 4) is that independently of the tumor coverage with a suitable absorbed dose the treatment efficacy was the same for a range of volume coverage comprised between 43 and 99.3% for 60Gy of minimum absorbed dose. This surprising observation implies an additional therapeutic effect than the one coming from the cytotoxicity effect delivered by Ho-166 ionizing radiation. This effect is thus coming from the combination of the microparticles present within the tumor that will participate in an additional immune modulation as will be observed in the immunohistochemistry section. The “positive fail pig” which received a tumor coverage of absorbed dose higher than some of the pigs of group 1 but failed to receive microparticles inside the tumor is a further indication that the additional effect that enhances the cytotoxicity of the ionizing radiations requires the presence of the microparticles inside the tumor.

TABLE 4
Tumor volume coverage for all the pigs treated with Ho-166 for two typical
values of absorbed dose, 60 and 100 Gy.

| | | Tumor | Tumor | |
|---|---|---|---|---|
| | | volume | volume | |
| | | coverage | coverage | |
| Groups | Pigs | at 60 Gy* | at 100 Gy | Status |
| Group 1 | 2 | 51.4% | 44.3% | >2 months survival |
| | 3 | 43% | 37.1% | >2 months survival |
| | 4 | 58.5% | 50.2% | >2 months survival |
| | 5 | 99.3% | 97.4% | >2 months survival |
| | 6 | 84.3% | 79.1% | >2 months survival |
| « Positive fail pig » | 1 | 55.5% | 49% | Euthanized |

*60 Gy is the typical target value used for external beam radiotherapy with a coverage of 98% of the tumor volume.

### Histology

Tumor was detected in all the animals of group 2 and control group. In the “positive fail pig”, the tumor was clearly observed. In group 1, a proper tumor was not identified in any of the 5 pigs due to severe necrosis and inflammation.

Holmium crystals were observed in almost all the animals of groups 1 and 2 (5/5 group 1, 3/3 group 2), but not in animals of control group. Holmium was visible as yellow-brown crystals, 30 μm in size with an irregular, angular shape, mainly located free in necrotic areas within the tumor.

In group 1, necrosis was severe, replacing the tumor, which was not identifiable any more in all the animals. It represented 60-100% of the lesion in all the animals, it was a liquefactive necrosis and it was associated to a granulomatous inflammation. The severity and the type of necrosis were indicative of a holmium-166 effect (Table 5). In contrast, necrosis was absent or mild (less than 30% of the lesion) in control or mild to moderate (30-60% of the lesion) in group 2.

Lymphocytic inflammation was slightly more severe in group 1 when compared to group 2 and control group (Table 5).

Granulomatous inflammation was evaluated on HE slides as the presence of recognizable macrophages, epithelioid cells and multinucleated giant cells. In group 1, a moderate to severe granulomatous reaction was observed (Table 5). It was located around necrotic areas, suggesting that it was an immune response to antigens liberated by holmium-induced necrosis. Holmium crystals themselves, independently from the radioactivity, probably participated in inducing this kind of inflammation, as a foreign body reaction, since a mild granulomatous reaction was also observed in group 2.

By contrast, inflammatory infiltrate was absent in all animals of the control group and mild in all animals of the group 2 (Table 5).

TABLE 5
Lesional score results of the findings observed on HE slides
from the brain of the animals of groups 1, 2 and control

| | “Positive | Group1 | | | | | Group 2 | | | Control | | |
|---|---|---|---|---|---|---|---|---|---|---|---|---|
| Finding | fail pig” | (Ho-166) | | | | | (Ho-165) | | | group | | |
| Animals | 1 | 2 | 3 | 4 | 5 | 6 | 7 | 8 | 9 | 10 | 11 | 12 |
| ¹Tumor | + | +/− | +/− | +/− | +/− | +/− | + | + | + | + | + | + |
| ¹Ho | − | + | + | + | + | + | + | + | + | − | − | − |
| ²Necrosis score | +++ | +++ | +++ | +++ | +++ | +++ | ++ | + | ++ | + | − | + |
| ³Lymphocytic | ++ | +++ | ++ | +++ | +++ | +++ | ++ | ++ | + | ++ | +++ | + |
| Inflammation | | | | | | | | | | | | |
| score | | | | | | | | | | | | |
| ³Granulomatous | − | ++ | +++ | ++ | +++ | + | + | + | + | − | − | − |
| Inflammation | | | | | | | | | | | | |
| score | | | | | | | | | | | | |

¹(+): present (+/−): not clearly visible ( − [symbol] absent
²(−) = absent, (+) = 1-30% of the tumor area, (++) = 31-60% of the tumor area, (+++) = 61-100% of the tumor area.
³(−) = absent, (+) = mild, (++) = moderate, (+++) = severe.

Immunohistochemistry

In this study, lymphocytic, macrophages and DCs infiltration were evaluated to characterize the immune microenvironment and try to better understand Holmium-166 mechanism of action. Immune surveillance implies natural killer and cytotoxic T lymphocytes (CTLs), both of them responsible of cancer cell death by cytotoxicity. Both of them are boosted by Interferon I, which is mainly secreted by plasmacytoid DCs. Conventional DCs, presenting the antigen by major histocompatibility (MHC) class II induce an adaptive response stimulating CD4T lymphocytes, and presenting the antigen by MHCI, stimulating CTLs (Frey B, et al. Immunol Rev. 2017; 280: 231-248.). These cells are necessary for developing an acquired specific immune response, which is particularly interesting when directed against tumoral cells. In addition, Tumor-associated macrophages (TAMs) which include pro-tumoral (M1 phenotype) and anti-tumoral (M2 phenotype) TAMs are important regulators of tumorigenesis.

Lymphocytes were characterized with anti-CD3 epsilon (T lymphocytes) and anti-Pax-5 (B lymphocytes) antibodies specific for swine lymphocytes as verified in control experiments. Plasmacytoid DCs were detected with the specific marker CD303. For macrophages and DCs identification, anti-Iba-1 was used as a panmarker recognizing both of the cell types, while anti-CD204 and anti-DC-Lamp were used to identify respectively macrophages and conventional DCs. In the positive control spleen, anti-Iba-1 antibody labelled splenic peri-arteriolar macrophages, reticular cells and T and B antigen presenting DCs, demonstrating its large spectrum in identifying macrophages/DCs in pigs. The used anti-CD204 antibody revealed to be specific for pig macrophages and the used anti-DC-lamp was specific for swine DCs but not macrophages.

Lymphocytic infiltration composed mainly of T lymphocytes was present in all the animals, without any remarkable difference among groups. CTLs or NK cells could not be identified since antibodies working on paraffin embedded pig tissues are not available. Nevertheless, the constant presence of lymphocytes among groups, independently from the holmium treatment, is indicative that they are not Holmium-166 treatment related.

Plasmacytoid DCs were present within all the brain lesions without any difference among groups, suggesting that their presence is related to the tumor, and not to a holmium effect. Their scant number indicates probably a not very prominent role in immune system tumoral microenvironment.

On the basis of the lack of differences in lymphocytic infiltration and plasmacytoid DC presence among the different groups, it was speculated that the observed necrosis in holmium-166 treated animals was not dependent on a cytotoxic lymphocytic activity, despite their presence.

In peritumoral location and around necrosis Iba-1+ macrophages/DCs were more abundant in groups 1 and 2 than in control group, indicating a holmium effect. Within the tumor, a large amount of Iba-1+ macrophages/DC were detected in all the animals of all the groups, indicating that the immune infiltrate is related to the presence of the tumor and not to a holmium effect. Within lymphocytic aggregates, no differences in the amount of Iba-1+ cells were observed among groups (Table 6).

In brain samples, CD204+ macrophages were more abundant in peritumoral location in groups 1 and 2 than in control group, indicating that their presence is associated to holmium treatment. Within the tumor and around necrosis, their presence was comparable in all the groups, indicating that CD204+ macrophages infiltrate in these locations is related to the presence of the tumor and not to a holmium effect (Table 6).

In brain samples, DC-lamp+ DCs were little in control group, slightly present in group 2 and abundant in group 1, indicating that holmium-166 is associated to a severe DC-lamp+ DC infiltration (Table 6).

In conclusion, from immunohistochemical analysis, in peritumoral location, a moderate to severe macrophage/DC infiltration was observed in groups 1 and 2, while it was slight in control group. This infiltrate was composed of macrophages and DCs, which were more abundant in group 1 than in group 2.

The intra-tumoral infiltrate was severe in all the groups; it was composed of macrophages but also DCs, especially in group 1. Necrosis was also associated to a moderate to severe macrophage/DC infiltrate, composed of macrophages in all the groups and also DCs in group 1. In all the groups, within lymphocytic aggregates, a moderate amount of macrophages/DCs were detected; they were composed mainly by DCs, though macrophages were also present.

Thus, holmium-166 was associated to a high infiltrate of conventional DCs around and inside the tumor; as well as around the necrosis and within the lymphocytic infiltrate.

DCs were almost absent in animals of group 2 and control group, while they were abundant around and inside the tumor and around the holmium associated necrosis. These cells are necessary for developing an acquired specific immune response, which is particularly interesting when directed against tumoral cells.

In addition to DCs, an increased number of macrophages were found in peritumoral location of holmium treated animals. Since it was observed in holmium-166 and holmium-165 treated animals, it was interpreted as a reaction to holmium particles or rather than to radioactivity only.

TABLE 6
Evaluation of macrophage and dendritic cells infiltration in the brain tumor

| | | | | | | | | Infiltrate around | | | Infiltrate around | | | Infiltrate within | | |
|---|---|---|---|---|---|---|---|---|---|---|---|---|---|---|---|---|
| | | Peritumoral | | | Intratumoral | | | coagulative | | | Ho associated | | | lymphocytic | | |
| | | infiltrate | | | infiltrate | | | necrosis | | | necrosis | | | aggregates | | |
| | | Iba- | CD | DC- | Iba- | CD | DC- | Iba- | CD | DC- | Iba- | CD | DC- | Iba- | CD | DC- |
| | Pigs | 1 | 204 | lamp | 1 | 204 | lamp | 1 | 204 | lamp | 1 | 204 | lamp | 1 | 204 | lamp |
| Positive | 1 | ++ | ++ | ++ | +++ | ++ | ++ | ++ | ++ | − | NA | NA | NA | +++ | − | + |
| fail pig | | | | | | | | | | | | | | | | |
| Group | 2 | + | − | ++ | ++ | ++ | − | NA | NA | NA | + | + | − | ++ | − | ++ |
| 1 | 3 | ++ | ++ | ++ | ++ | +++ | +++ | NA | NA | NA | ++ | +++ | ++ | ++ | − | + |
| | 4 | ++ | − | +++ | +++ | ++ | +++ | NA | NA | NA | ++ | + | +++ | +++ | + | +++ |
| | 5 | +++ | ++ | +++ | +++ | ++ | ++ | NA | NA | NA | +++ | ++ | ++ | +++ | − | ++ |
| | 6 | ++ | +++ | +++ | ++ | +++ | ++ | NA | NA | NA | ++ | ++ | ++ | ++ | + | ++ |
| Group | 7 | +++ | + | ++ | +++ | ++ | − | +++ | +++ | − | +++ | ++ | − | +++ | + | + |
| 2 | 8 | +++ | ++ | + | +++ | ++ | − | +++ | ++ | − | ++ | +++ | +++ | +++ | + | + |
| | 9 | +++ | ++ | + | +++ | ++ | − | ++ | ++ | − | +++ | ++ | − | ++ | − | ++ |
| Control | 10 | − | − | − | +++ | ++ | + | + | + | − | NA | NA | NA | ++ | − | + |
| group | 11 | + | − | + | ++ | ++ | + | NA | NA | NA | NA | NA | NA | ++ | − | ++ |
| | 12 | + | + | + | ++ | ++ | ++ | ++ | ++ | + | NA | NA | NA | + | + | ++ |

(−) = absence of labelled cells, (+) = mild amount of labelled cells, (++) = moderate amount of labelled cells, (+++) = severe amount of labelled cells.

### CONCLUSION

This trial demonstrated the excellent tumor control efficiency as well as the absence of toxic effects of Ho-166/Ho-165 microbrachytherapy in a human glioblastoma model in minipigs, using one of the most radioresistant cell lines (U87). In view of these encouraging results, intratumoral treatment with Ho-166/Ho-165 microparticles could be considered as a promising therapeutic approach for the treatment of glioblastoma as well as of other solid tumors.

The antitumoral effect observed cannot be explained by a single effect of the ionizing radiation since the percentage of the tumor volume which received more than 60Gy or 100Gy (considered as the filling dose into the literature) is not correlated with the response (decrease of the tumor volume). These results show that besides the direct effect of the ionizing radiation the microparticles delivered inside the tumor induced anti-tumor immunity mechanisms by the recruitment of external macrophages and dendritic cells and activate the production of T cells lymphocytes. This last response is prolonged due to the persistency of the microparticles (as stable implants) in the tumors tissue. Thereby, the localized antitumor effect induced by the combination of radiotherapy and direct TAMs toxicity can generate an abscopal effect extended to the total tumor mass plus its surrounding infiltration area.

Another advantage of this novel Ho-166/Ho-165 microbrachytherapy in comparison with other treatment modalities is the single session approach and its minimally invasive nature, without the severe secondary effects that are often encountered with chemotherapy and other radiotherapy methods. In particular the flexibility observed in terms of dosimetry is totally opposite to the requirements existing in External Beam Radiotherapy (EBRT) demonstrating here an innovating new therapy modality which open the way to better treatment efficacy.

1. A method for treating cancer by combined antitumoral effect of radioactivity and intratumoral microparticle deposition, the method comprising administering, by intratumoral injection, a composition comprising (1) microparticles, the microparticles comprising a radioactive isotope suitable for use in internal radiotherapy, and (2) an injection vehicle.

2. The method for treating cancer according to claim 1, wherein an absorbed dose superior or equal to 60 Grays is administered in less than 95% of the tumor volume.

3. The method for treating cancer according to claim 1, wherein an absorbed dose superior or equal to 100 Grays is administered in less than 95% of the tumor volume.

4. The method for treating cancer according to claim 1, wherein intratumoral microparticle deposition induces a depletion of pro-tumoral macrophages from the treated tumor.

5. The method for treating cancer according to claim 1, wherein intratumoral microparticle deposition induces an anti-tumor immune response.

6. The method for treating cancer according to claim 1, wherein the radioactive isotope is selected from the group comprising: holmium-166, lutetium-177, rhenium-186 or -188, gold-198, yttrium-90, thorium-227, actinium-225 and combinations thereof.

7. The method for treating cancer according to claim 1, wherein the microparticles further comprise a non-radioactive isotope possibly from the same chemical element.

8. The method for treating cancer according to claim 7, wherein the microparticles comprise holmium-165 and holmium-166.

9. The method for treating cancer according to claim 1, wherein the microparticles comprise or consist of polysiloxane, acetlyacetonate (AcAc), Poly L-Lactic Acid (PLLA), alginate, chitosan, resin, glass and combination thereof.

10. The method for treating cancer according to claim 1, wherein the microparticles have a mean or median size from 0.1 μm to 10 μm.

11. The method for treating cancer according to claim 1, wherein the microparticles have a specific activity of at least 1 megabecquerel (MBq) per milligram.

12. The method for treating cancer according to claim 1, wherein the composition comprises a microparticle suspension having a density of at least 1 g/mL.

13. The method for treating cancer according to claim 1, wherein the composition comprises a microparticle suspension having a viscosity of at least 3 mPa·s.

14. The method for treating cancer according to claim 1, wherein the composition comprises an aqueous suspension of the radioactive microparticles in an injection vehicle comprising water and eventually less than 5% (v/v) of ethanol.

15. The method for treating cancer according to claim 1, wherein the cancer is selected from the group comprising: bladder, brain, breast, colon, gastric, eye, head and neck, skin, kidney, liver, lung, ovary, oral, prostate, small intestine, soft-tissue, testis, thyroid, and uterus cancer.

16. The method for treating cancer according to claim 1, wherein the composition is administered by intratumoral injection at a sub-optimal tumor volume coverage dose of radioactivity.

17. The method for treating cancer according to claim 1, wherein an absorbed dose superior or equal to 60 Grays is administered in 35% to 55% of the tumor volume.

18. The method for treating cancer according to claim 1, wherein an absorbed dose superior or equal to 100 Grays is administered in 35% to 55% of the tumor volume.

19. The method for treating cancer according to claim 7, wherein the microparticles comprise holmium-165 and holmium-166, and at least 20% (w/w) of total holmium.

20. The method for treating cancer according to claim 1, wherein the cancer is glioblastoma or metastatic brain tumor.
